(19) United States
(12) Patent Application Publication
(10) Pub. No.: US 2025/0027752 A1
(43) Pub. Date: Jan. 23, 2025

(54) Non-lethal Jellyfish Composition Spray

(71) Applicant: Tim Joyner, Mountain View, AR (US)

(72) Inventor: Tim Joyner, Mountain View, AR (US)

(21) Appl. No.: 18/225,149

(22) Filed: Jul. 23, 2023

(51) Int. Cl. F41H 9/10; A61K 35/614

(52) U.S. Cl. CPC F41H 9/10; A61K 35/614

(57) ABSTRACT

A non-lethal spray composition comprising jellyfish venom for use in military or civilian applications. The spray composition comprises a mixture of jellyfish venom, a propellant, a solvent, stabilizers, and agents to prevent bacterial and fungal growth. The composition provides a safe and effective means of incapacitating individuals without causing permanent harm.

### PRIOR ART

While there are many methods of neutralizing venoms and toxins such as U.S. Pat. Nos. 9,616,095, 11,352,393, and 2017/0119817, there has never been an application which uses some of these venoms as a means incapacitation or crowd control.

### BACKGROUND OF THE INVENTION

Military and law enforcement personnel require non-lethal means of controlling hostile individuals in situations where lethal force is not warranted. Pepper spray is commonly used for this purpose, but it can cause permanent harm or even death in individuals with respiratory conditions or allergies. There is a need for a non-lethal spray that can effectively incapacitate individuals without causing permanent harm.

The non-lethal spray composition comprising jellyfish venom offers several advantages over existing non-lethal sprays. The venom is non-lethal and can be obtained from jellyfish species that are not endangered or made in a laboratory under controlled conditions.

### SUMMARY OF THE INVENTION

In the following description, numerous specific details are set forth to clearly describe various specific embodiments disclosed herein. One skilled in the art, however, will understand that the presently claimed invention may be practiced without all of the specific details discussed below. In other instances, well known features have not been described so as not to obscure the invention.

The present subject matter provides a non-lethal spray composition comprising jellyfish venom for use in military or civilian applications. The composition comprises a mixture of jellyfish venom, a propellant, a solvent, stabilizers, and agents to prevent bacterial and fungal growth.

The jellyfish venom is obtained from jellyfish species that are known to have non-lethal venom. The venom is purified to remove any toxic components and is then mixed with the other ingredients to form a stable and effective spray composition.

In some embodiments, the venom can be synthesized and made synthetically. To make synthetic jellyfish venom, the amino acid sequence of the venom protein is determined, and the protein is then synthesized using recombinant DNA technology. The synthetic protein is then purified, and its structure is confirmed using analytical techniques like NMR spectroscopy. The synthetic jellyfish venom is then mixed with the other ingredients to form a stable and effective spray composition.

The propellant used in the composition can be any suitable gas, such as nitrogen, carbon dioxide, or compressed air. The solvent used can comprise of any suitable organic solvent, such as ethanol, isopropanol, dimethyl sulfoxide (DMSO) or acetone.

The stabilizers used can comprise of any suitable material, such as sodium benzoate, potassium sorbate, sodium metabisulfite or ascorbic acid. The agents used to prevent bacterial and fungal growth can be any suitable material, such as benzalkonium chloride, chlorhexidine gluconate, or triclosan.

In some embodiments, the non-lethal spray composition is packaged in a canister similar to that used for pepper spray. The canister can be pressurized to allow for easy and effective deployment of the spray. The spray can be directed at the face or body of the individual to be incapacitated, causing temporary pain and discomfort. The composition can be formulated to deliver a fine mist or a stream of spray, depending on the desired application and range.

In other embodiments, the non-lethal spray composition can be incorporated into other devices, such as stun grenades, paintball pellets, or projectiles fired from a launcher, to provide additional options for deployment and facilitate use in various tactical situations.

The non-lethal jellyfish venom spray composition can effectively incapacitate individuals without causing permanent harm or posing a significant risk to individuals with respiratory conditions or allergies. The spray may cause temporary discomfort, such as pain, itching, or irritation, but these effects are generally short-lived and can be treated with standard first aid measures.

In some embodiments, an antidote or neutralizing agent may be included with the non-lethal spray composition or provided separately for use by law enforcement or military personnel. The antidote can be formulated as a topical cream, gel, or spray that can be applied to the affected area to alleviate symptoms and accelerate recovery. Suitable antidotes may include antihistamines, analgesics, anti-inflammatory agents, or other compounds known to reduce the effects of jellyfish venom.

### BRIEF DESCRIPTION OF DRAWINGS

The accompanying drawings, which are incorporated in and constitute a part of this specification, illustrate various embodiments of the invention.

FIG. 1 is a perspective view of a canister containing the non-lethal jellyfish venom spray composition according to one embodiment.

FIG. 2 is a cutaway view of a canister containing the non-lethal jellyfish venom spray composition according to one embodiment.

### DETAILED DESCRIPTION OF THE INVENTION

The present invention relates to a non-lethal spray composition comprising jellyfish venom for use in military or civilian applications. The composition provides a safe and effective means of incapacitating individuals without causing permanent harm or posing significant risks to individuals with respiratory conditions or allergies.

While the present invention has been described in connection with various embodiments, it will be understood that the invention is not limited to those embodiments. On the contrary, the invention is intended to cover all alternatives, modifications, and equivalents, as may be included within the spirit and scope of the appended claims. In addition to the embodiments described above, the non-lethal jellyfish venom spray composition may also be provided with additional features or enhancements to improve its usability, safety, and effectiveness. Some potential additions and improvements include:

Adjustable spray patterns: The spray nozzle or delivery mechanism can be designed to allow the user to adjust the spray pattern between a fine mist, stream, or foam to adapt to different situations and distances.

Dye markers: The composition may include a visible or UV-reactive dye to mark the individuals exposed to the spray for later identification or tracking purposes.

Safety measures: The canister or delivery device may be equipped with safety features, such as a safety lock or cap, to prevent accidental discharge of the spray during transportation or storage.

Training formulations: A non-venomous, inert formulation of the spray may be developed for use in training exercises, allowing law enforcement and military personnel to practice and familiarize themselves with the use of the non-lethal jellyfish venom spray without the risk of injury or harm.

Environmental considerations: Efforts can be made to minimize the environmental impact of the spray composition by using biodegradable materials, reducing or eliminating the use of hazardous substances, and implementing sustainable sourcing and manufacturing practices.

Integration with existing equipment: The non-lethal jellyfish venom spray composition can be designed to be compatible with existing devices and equipment used by law enforcement and military personnel, such as holsters, tactical vests, and weapon systems, to facilitate seamless integration into their existing gear.

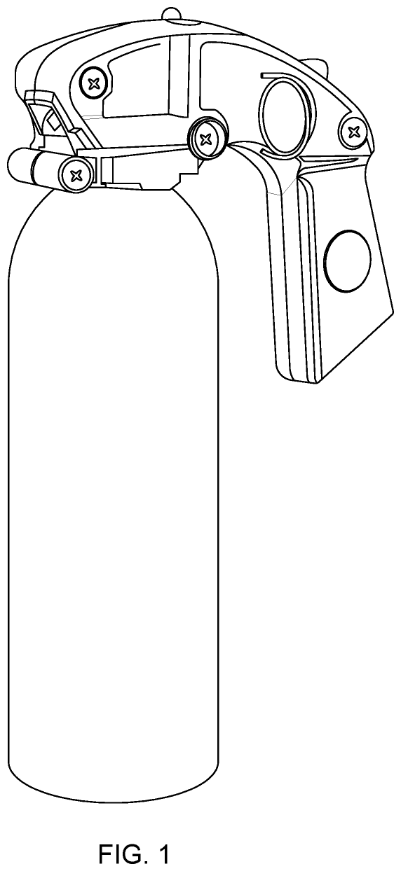

FIG. 1 shows a perspective view of a canister containing the non-lethal jellyfish venom spray composition.

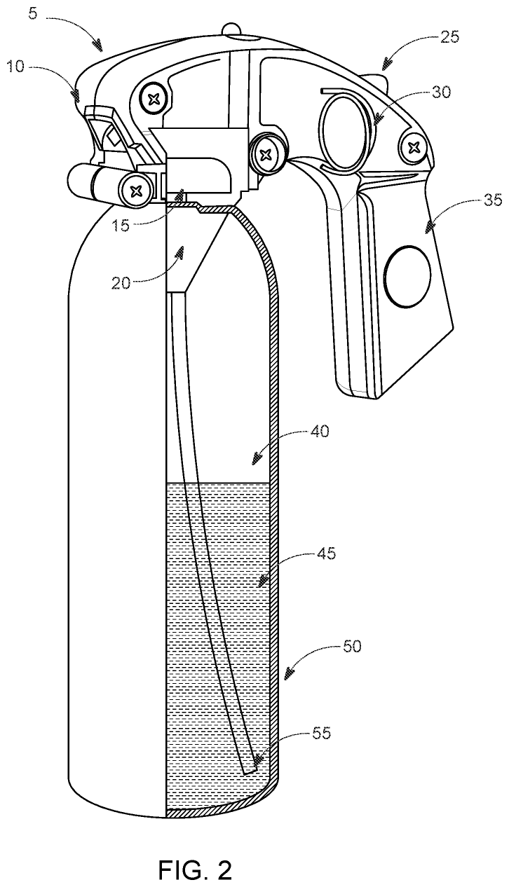

FIG. 2 shows a perspective view of a canister containing the non-lethal jellyfish venom spray composition wherein the cover 5 protects the internal mechanism of the jellyfish spray composition. The nozzle 10 directs the jellyfish spray composition when in use. The actuator 15 allows the valve 20 to pass the jellyfish spray composition out through the nozzle. The propellant 40 forces the liquid formula 45 through the dip tube 55 and out of the canister 50.

The safety pin 30 prevents the accidental discharge of the non-lethal jellyfish spray composition. The handle 35 allows the user to grip the container and activate the spray actuator 25 which then causes the discharge of the non-lethal jellyfish spray composition.

These additional features and enhancements are provided as examples of potential improvements to the non-lethal jellyfish venom spray composition and should not be construed as limiting the scope of the invention. The invention is intended to cover all alternatives, modifications, and equivalents within the spirit and scope of the appended claims.

### LIST OF ELEMENTS OF THE FIGURES

5—Cover
10—Nozzle
15—Actuator
20—Valve
25—Spray actuator
30—Safety pin
35—Handle
40—Propellant
45—Liquid formula
50—Canister
55—Dip tube

1. A non-lethal jellyfish composition spray comprising of a jellyfish venom, a propellant, a solvent, stabilizers, anti-bacterial agents, and anti-fungal agents.

2. The non-lethal jellyfish composition spray comprising of the jellyfish venom, the propellant, the solvent, stabilizers, anti-bacterial agents, and anti-fungal agents as in claim 1, wherein the jellyfish venom is obtained from jellyfish species known to have non-lethal venom.

3. The non-lethal jellyfish composition spray comprising of the jellyfish venom, the propellant, the solvent, stabilizers, anti-bacterial agents, and anti-fungal agents as in claim 1, wherein the jellyfish venom is purified to remove any toxic components.

4. The non-lethal jellyfish composition spray comprising of the jellyfish venom, the propellant, the solvent, stabilizers, anti-bacterial agents, and anti-fungal agents as in claim 1, wherein the jellyfish venom may also be synthesized.

5. The non-lethal jellyfish composition spray comprising of the jellyfish venom, the propellant, the solvent, stabilizers, anti-bacterial agents, and anti-fungal agents as in claim 1, wherein the propellant further comprises of nitrogen, carbon dioxide, or compressed air.

6. The non-lethal jellyfish composition spray comprising of the jellyfish venom, the propellant, the solvent, stabilizers, anti-bacterial agents, and anti-fungal agents as in claim 1, wherein the solvent further comprises of ethanol, isopropanol, dimethyl sulfoxide (DMSO) or acetone.

7. The non-lethal jellyfish composition spray comprising of the jellyfish venom, the propellant, the solvent, stabilizers, anti-bacterial agents, and anti-fungal agents as in claim 1, wherein the stabilizers further comprises of sodium benzoate, potassium sorbate, sodium metabisulfite, or ascorbic acid.

8. The non-lethal jellyfish composition spray comprising of the jellyfish venom, the propellant, the solvent, stabilizers, anti-bacterial agents, and anti-fungal agents as in claim 1, wherein anti-bacterial agents and anti-fungal agents further comprises of benzalkonium chloride, chlorhexidine gluconate, or triclosan.

9. A method of manufacturing a non-lethal jellyfish composition spray comprising of a jellyfish venom, a propellant, a solvent, stabilizers, anti-bacterial agents, and anti-fungal agents.

10. The method of manufacturing the non-lethal jellyfish composition spray comprising of the jellyfish venom, the propellant, the solvent, stabilizers, anti-bacterial agents, and anti-fungal agents as in claim 9, wherein the jellyfish venom is obtained from jellyfish species known to have non-lethal venom.

11. The method of manufacturing the non-lethal jellyfish composition spray comprising of the jellyfish venom, the propellant, the solvent, stabilizers, anti-bacterial agents, and anti-fungal agents as in claim 9, wherein the jellyfish venom is purified to remove any toxic components.

12. The method of manufacturing the non-lethal jellyfish composition spray comprising of the jellyfish venom, the propellant, the solvent, stabilizers, anti-bacterial agents, and anti-fungal agents as in claim 9, wherein the jellyfish venom may also be synthesized.

13. The method of manufacturing the non-lethal jellyfish composition spray comprising of the jellyfish venom, the propellant, the solvent, stabilizers, anti-bacterial agents, and anti-fungal agents as in claim 9, wherein the propellant further comprises of nitrogen, carbon dioxide, or compressed air.

14. The method of manufacturing the non-lethal jellyfish composition spray comprising of the jellyfish venom, the propellant, the solvent, stabilizers, anti-bacterial agents, and anti-fungal agents as in claim 9, wherein the solvent further comprises of ethanol, isopropanol, dimethyl sulfoxide (DMSO) or acetone.

15. The method of manufacturing the non-lethal jellyfish composition spray comprising of the jellyfish venom, the propellant, the solvent, stabilizers, anti-bacterial agents, and anti-fungal agents as in claim 9, wherein the stabilizers further comprises of sodium benzoate, potassium sorbate, sodium metabisulfite, or ascorbic acid.

16. The method of manufacturing the non-lethal jellyfish composition spray comprising of the jellyfish venom, the propellant, the solvent, stabilizers, anti-bacterial agents, and anti-fungal agents as in claim 9, wherein anti-bacterial agents and anti-fungal agents further comprises of benzalkonium chloride, chlorhexidine gluconate, or triclosan.
